(19) United States
(12) Patent Application Publication
(10) Pub. No.: US 2012/0290010 A1
(43) Pub. Date: Nov. 15, 2012

(54) Flexible Screw Head Constructs for Spinal Stabilization

(75) Inventors: Shahram Shaun Zamani, Poway, CA (US); Nicholas M. Cordaro, Vista, CA (US)

(73) Assignee: Seaspine, Inc., Vista, CA (US)

(21) Appl. No.: 13/510,451

(22) PCT Filed: Nov. 17, 2010
(86) PCT No.: PCT/US10/57076

§ 371 Date: Aug. 3, 2012

Related U.S. Application Data
(60) Provisional application No. 61/262,477, filed on Nov. 18, 2009.

(51) Int. Cl. A61B 17/70

(52) U.S. Cl. 606/264

(57) ABSTRACT

Devices and systems related to an improved spinal pedicle based construct are disclosed herein along with the related method of manufacturing and operation thereof. The improved spinal pedicle based construct may include a flexible membrane(s) or mechanism(s) incorporated between the anchoring component and the longitudinal membrane. The flexible membrane(s) incorporated may provide controlled motion of the functional spine unit connected to the anchoring component with respect to the posterior construct. Moreover, when the patient moves his or her back, the flexible membrane(s) may also divert a force away from the anchoring component, thereby reducing the chances of bone cracking.

### CROSS-REFERENCE TO RELATED APPLICATION

This application claims priority from Provisional Application No. 61/262,477 filed on Nov. 18, 2009.

### BACKGROUND OF THE INVENTION

1. Field of the Invention.

This application relates to devices, systems and methods directed to flexible spinal fixation allowing stabilization of a spine.

2. Description of Related Art.

Stabilization of the spine is an important aspect of spinal surgery procedures and is a significant factor in the healing process for patients. One stabilization technique currently practiced is to drill screws into the bone of the patient spine and utilize one or more rods to hold the screws together to fashion a “frame” for holding the vertebrae still.

Unfortunately, traditional screw and rod systems do not allow for movement of the patient's back. By limiting the patient's movement, substantial discomfort may arise, and in an effort to alleviate such discomfort, some patients may attempt to move their backs. Such movement may cause the screws and rods to loosen, ultimately leading to further damage to the patient's vertebrae.

Accordingly, certain advancements have introduced dynamic rods that may have a slight flex to them to allow limited back movement. Other advancements have introduced a pivoting screw head to achieve the same. However, these advancements now suffer from the problem of allowing too much motion of the patient's back, which may lead to unnecessarily prolonged recovery times. Moreover, these systems also channel the force created when the patient moves onto the screw that is attached to the bone of the vertebrae, thereby increasing the chances that the bone will crack. This is especially problematic in patients with osteoporosis or other bone-weakening conditions.

What is needed is a spine stabilization system that allows controlled movement without placing unnecessary strain on the backbones.

### SUMMARY OF THE INVENTION

An improved spinal pedicle based construct, in accordance with this disclosure, includes a flexible membrane or mechanism incorporated between the anchoring component and the longitudinal membrane. The flexible membrane incorporated may provide controlled motion of the functional spine unit connected to the anchoring component with respect to the posterior construct. Moreover, the flexible membrane may also divert a force created when the patient moves away from the anchoring component, thereby reducing the chances of bone cracking.

In one embodiment, a spinal fixation device comprises a head component having an interior cavity, a first flexible membrane housed inside the head component, and a screw, wherein a first end of the screw is fixable to a pedicle bone of a patient's spine and a second end is attached to the first flexible membrane, wherein the first flexible membrane allows the head component to move in a first direction while keeping the first end of the screw fixed to the pedicle bone. The spinal fixation device may further comprise a second flexible membrane housed inside the head component, wherein the second flexible membrane is configured to allow the head component to move in a second direction. The spinal fixation device may further comprise a third flexible membrane housed inside the head component, wherein the third flexible membrane is configured to allow the head component to move in a second direction. The spinal fixation device may further include the characteristic of wherein the head component is configured to move in a third direction. The spinal fixation device may further include the characteristic of wherein the first direction is away from a screw axis, the second direction is along the screw axis, and the third direction is torsioning about the screw axis. The spinal fixation device may further include the characteristic of wherein the head component is configured to move a first distance in the first direction when a first force is applied. The spinal fixation device may further include the characteristic of wherein the head component is configured to move a second distance in the first direction when the first force applied is increased, wherein the force required to move the head component the second distance is proportionally greater than the force required to move the head component the first distance. The spinal fixation device may further include the characteristic of wherein the head component is configured to move a first distance in the third direction when a first force is applied. The spinal fixation device may further include the characteristic of wherein the head component is configured to move a second distance in the third direction when the first force applied is increased, wherein the force required to move the head component the second distance is proportionally greater than the force required to move the head component the first distance. The spinal fixation device may further include the characteristic of wherein the first flexible membrane is configured to a membrane selected from a group of membranes, each membrane of the group of membranes having a different flexibility, further wherein the flexibility of the membrane selected determines the flexibility of the spinal fixation device.

In another embodiment, a spinal fixation device may include a flexible component, the flexible component having a geometry and constructed out of at least one material wherein the geometry of the flexible component and the material of the flexible component determines the flexibility of the spinal fixation device. The spinal fixation device may further include a frame configured to fit within a tulip head of the spinal fixation device, one or more points of attachment connecting the frame to an inner circumference, and wherein the inner circumference configured to hold a screw attached to a pedicle bone, and further wherein a movement of the inner circumference allows the tulip head to bend with respect to a screw head when a force up to a threshold is exerted on the tulip head by the rod. The spinal fixation device may further include the characteristic of wherein the movement of the inner circumference continues to allow the tulip head to bend with respect to the screw head when a force beyond the threshold is exerted on the head by the rod, further wherein a degree of further bending of the tulip head is reduced when the force exerted is beyond the threshold. The spinal fixation device may further include the characteristic of wherein the flexible component is constructed out of a polymer selected from a group consisting of: PEEK, PCU, polyester and polyethylene. The spinal fixation device may further include the characteristic of wherein the flexible component incorporates a material selected from a group consisting of: titanium, cobalt chromium, steel and nitinol. The spinal fixation device may further include the characteristic of wherein the flexible component is constructed out of a polymer selected from a group consisting of: PEEK, PCU, polyester and polyethylene and further incorporates a material selected from a group consisting of: titanium, cobalt chromium, steel and nitinol.

In another embodiment, a spinal fixation system may comprise a first spinal fixation device, a second spinal fixation device, and a rod connecting the first spinal fixation device to the second spinal fixation device, wherein at least one of the first and second spinal fixation device includes a flexible head component having a flexible membrane. The spinal fixation system may further include the characteristic of wherein the first spinal fixation device is a pseudo polyscrew device and the second spinal fixation device is a complete polyscrew device. The spinal fixation system may further include the characteristic of wherein the first and second spinal fixation device are pseudo polyscrew devices and the rod is a non-flexible rod. The spinal fixation system may further include the characteristic of wherein the first spinal fixation device includes a first flexible membrane having a first number of points of attachment to a frame of the first flexible membrane and wherein the second spinal fixation device includes a second flexible membrane having a second number of points of attachment to a frame of the second flexible membrane. The spinal fixation system may further include the characteristic of wherein the first flexible membrane is more flexible than the second flexible membrane.

In another embodiment, the spinal-based construct includes a tulip head for housing the flexible membrane, and for attaching the screw and rod. The tulip head may be constructed out of one or more components, and the flexible membrane may allow the tulip head to controllably move about a screw axis, and bend away from the screw axis, thereby allowing flexibility. In one aspect, the screw axis may be defined by the screw. That is, the axis may be the line drawn between the tip of the screw and the head of the screw. The screw may attach to the tulip head at one end, while the rod may be received in an adjacent direction to the screw attachment direction at a different portion of the tulip head.

In another embodiment, the spinal-based construct includes a tulip head for housing several flexible membranes, and for attaching the screw and rod. Under this embodiment, a first or primary flexible membrane may be placed between two additional flexible membranes within the tulip head. The additional flexible membranes may provide controlled motion along the screw axis. The tulip head may be constructed out of one or more components, and the primary flexible membrane may allow the tulip head to controllably flex away from the screw axis and return to a position substantially located on the screw axis, thereby allowing flexibility. The screw may attach to the tulip head at one end, while the rod may be received in a substantially perpendicular direction to the screw attachment direction at a different portion of the tulip head.

In another embodiment, the spinal based construct may be a side-loading polyscrew system with a flexible membrane located between the screw and the tulip head. The rod may be offset from the location of the attachment between the screw and the tulip head, and the direction of the rod may be orthogonal to the direction of the screw.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings.

FIG. 1A is a perspective view of a screw and rod spinal construct, in accordance with one or more embodiments described herein;

FIG. 1B is a perspective view of a screw and rod spinal construct, with the head component in a displaced state in accordance with one or more embodiments described herein;

FIG. 1C is a partially deconstructive view of the construct of FIG. IA in accordance with one or more embodiments described herein;

FIG. 2A is an exploded view of the unassembled construct of FIG. 1C in accordance with one or more embodiments described herein;

FIG. 2B is an view of the manufacturing set up for the unassembled construct of FIG. 2A in accordance with one or more embodiments described herein;

FIG. 3 is an exploded view of an unassembled construct in accordance with one or more embodiments described herein;

FIG. 4 is an enlarged view of one of the flexible components shown in FIG. 2 in accordance with one or more embodiments described herein;

FIG. 5 is an enlarged view of an alternative flexible component in accordance with one or more embodiments described herein;

FIG. 6 is an exploded view of an unassembled construct in accordance with one or more embodiments described herein;

FIG. 7A is a perspective view of a screw and rod spinal construct, in accordance with one or more embodiments described herein;

FIG. 7B is an exploded view of the unassembled construct of FIG. 7A in accordance with one or more embodiments described herein;

FIG. 8A is a perspective view of a screw and rod spinal construct, in accordance with one or more embodiments described herein;

FIG. 8B is an exploded view of the unassembled construct of FIG. 8A in accordance with one or more embodiments described herein;

FIG. 9A is a graph depicting a force-displacement relationship for a prior art screw and rod construct;

FIG. 9B is a graph depicting a force-displacement relationship for a prior art screw and rod construct;

FIG. 9C is a graph depicting a force-displacement relationship for a screw and rod construct in accordance with one or more embodiments described herein;

FIG. 9D is a graph depicting a force-displacement relationship for a screw and rod construct in accordance with one or more embodiments described herein;

FIG. 9E is a graph depicting a force-displacement relationship for a screw and rod construct in accordance with one or more embodiments described herein;

FIG. 9F is a graph depicting a force-displacement relationship for a screw and rod construct in accordance with one or more embodiments described herein;

FIG. 9G is a graph depicting an angle-moment relationship for a screw and rod construct in accordance with one or more embodiments described herein;

FIG. 9H is a graph depicting an angle-moment relationship for a screw and rod construct in accordance with one or more embodiments described herein;

FIG. 9I is a graph depicting an angle-moment relationship for a screw and rod construct in accordance with one or more embodiments described herein;

FIG. 9J is a graph depicting an angle-moment relationship for a screw and rod construct in accordance with one or more embodiments described herein;

FIG. 10A is a graph depicting a force-displacement relationship for a screw and rod construct in accordance with one or more embodiments described herein;

FIG. 10B is a graph depicting a force-displacement relationship for a screw and rod construct in accordance with one or more embodiments described herein;

FIG. 10C is a graph depicting a force-displacement relationship for a screw and rod construct in accordance with one or more embodiments described herein; and

FIG. 10D is a graph depicting a force-displacement relationship for a screw and rod construct in accordance with one or more embodiments described herein.

### DETAILED DESCRIPTION OF THE PREFERRED EMBODIMENTS

Persons skilled in the art will readily appreciate that various aspects of the disclosure may be realized by any number of methods and devices configured to perform the intended functions. Stated differently, other methods and devices may be incorporated herein to perform the intended functions. It should also be noted that the drawing FIGS. referred to herein are not all drawn to scale, but may be exaggerated to illustrate various aspects of the invention, and in that regard, the drawing FIGS. should not be construed as limiting. Finally, although the present disclosure may be described in connection with various medical principles and beliefs, the present disclosure should not be bound by theory.

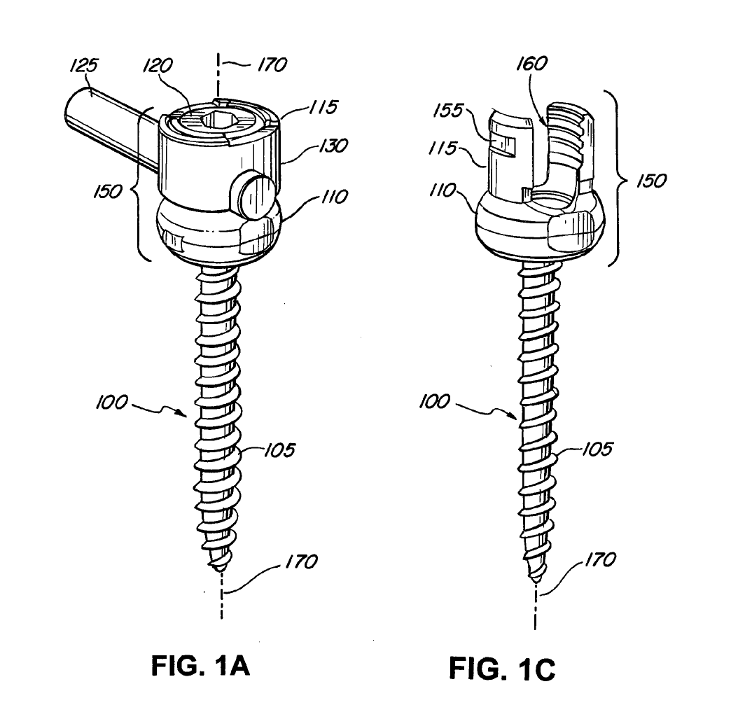

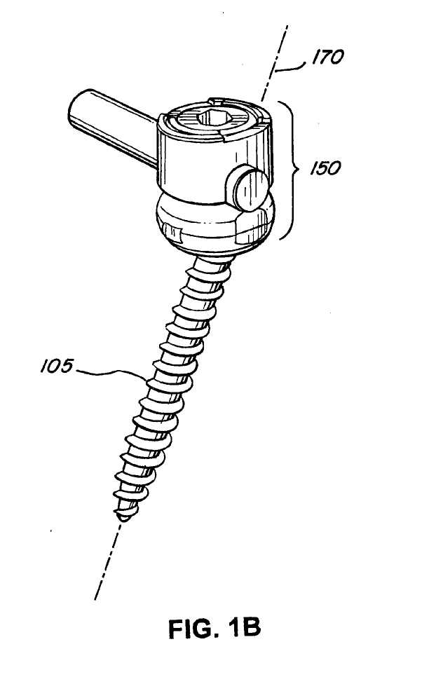

Referring now to FIG. 1A, a flexible spinal fixation assembly 100 is shown. The spinal fixation assembly 100 may be a posterior pedicle mono/poly screw that is fixably anchored to a functional spinal unit with bone screw 105 and connected to an adjacent spinal fixation assembly with rod 125. In one aspect, the adjacent spinal fixation assembly connected by rod 125 may be the same flexible spinal fixation assembly 100 or it may be a different fixation assembly. As shown in FIG. 1A, the spinal fixation assembly 100 further includes a head component 150 comprising a screw attachment portion 110 and rod insertion portion 115. The head component 150 may further include a bolt 120 for holding the rod 125 in place. The head component 150 may also include a cap 130 which may include a locking member (not shown) for fitting in slot 155 (see FIG. 1B) and holding the cap 130 in place. As shown, FIG. 1A is in a non-displaced state where the screw 105 and the head component 150 both lie on the screw axis 170. FIG. 1B illustrates the head component 150 in a displaced state when a pressure is exerted on the head component 150. Here, the head component 150 no longer lies on the screw axis 170 but is displaced or flexibly bent away from the screw axis 170. Once the force is removed, the head component 150 may move back towards the screw axis 170 and may revert back to the position shown in FIG. 1A. In one aspect, some of the components such as the bolt, cap, screw, screw attachment portion and rod insertion portion may be constructed solely out of, or alternatively incorporate a material such as titanium, cobalt chromium, steel and nitinol.

FIG. 1C is the flexible spinal fixation assembly 100 of FIG. 1A without the rod 125, the bolt 120 and the cap 130. These portions of the flexible spinal fixation assembly 100 have been removed in FIG. 1C to better illustrate that the rod 125 may be inserted during construction between grooves 160 and the screw attachment portion 110. The cap 130 may be attached after rod 125 is inserted and held in place by slot 155. Additionally, bolt 120 may be received and tightened to be held in place by grooves 160.

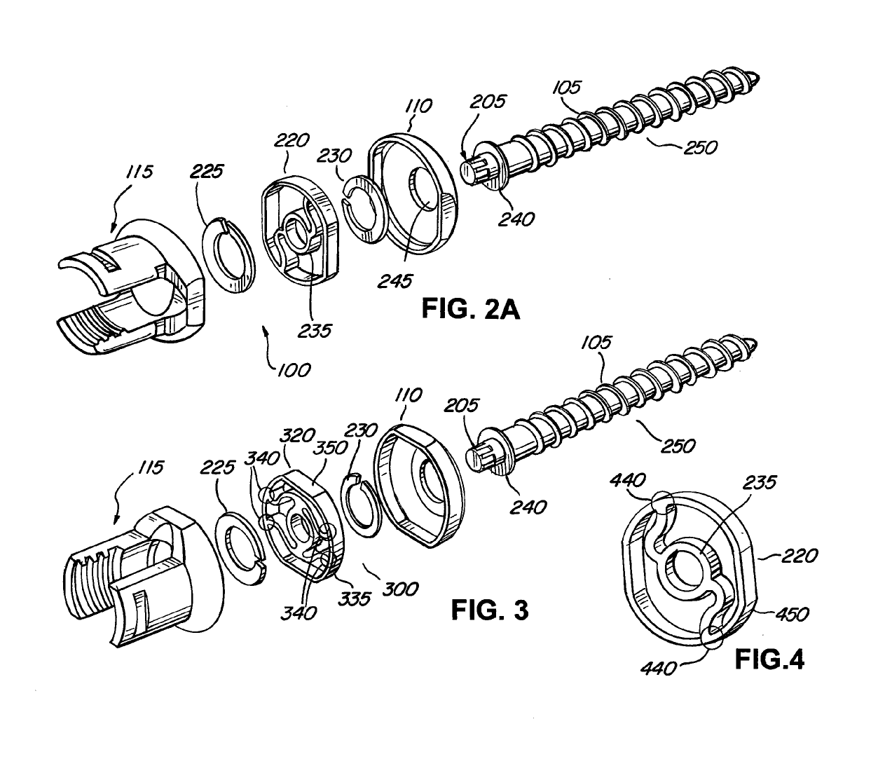

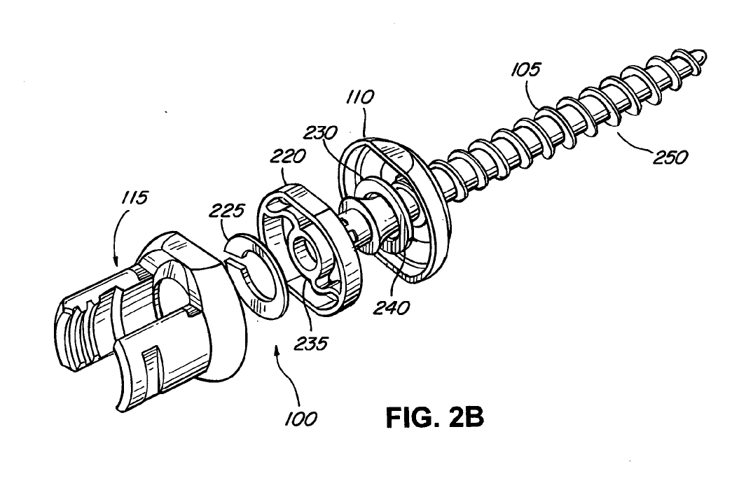

FIG. 2A is an exploded view of the flexible spinal fixation assembly 100 of FIGS. 1A and 1C. In other words, FIG. 2A may depict a further deconstructed flexible spinal fixation assembly 100 of FIGS. 1A and 1C. The head component 150, shown in FIG. 1C to include rod insertion portion 115 and screw attachment portion 110, is deconstructed in FIG. 2A to further include a flexible membrane 220 and accompanying flexible membranes 225 and 230. The flexible membrane 220 may control the degree of flexibility or radial displacement of the head component 150 based on the design and elastic properties of the flexible membrane 235. That is, the more elastic flexible membrane 220 is, the higher the degree of angular displacement of the head component 150 with respect to the screw axis (e.g., screw axis 170 of FIG. 1A). Here, the inner circumference 235 may be configured to tightly fit over the head portion 205 of the screw 105 and hold the head portion 205 in place. However, while the head portion 205 of the screw 105 is held in place by the inner circumference 235, the head component 150 may flex and displace orthogonally with respect to the screw axis when a force is applied (as shown in FIG. 1B) and may return to a position substantially perpendicular to the direction of the screw (as shown in FIG. 1A and 1C) when a force is not applied or removed. The flexible membranes 225 and 230 serve to allow the head component 150 to move along the screw axis and results in the flexible spinal fixation assembly 100 to slightly compress or elongate along the screw axis when a force substantially along the direction of the screw axis is exerted on the flexible spinal fixation assembly 100.

FIG. 2B illustrates a manufacturing set-up of the assembly of FIG. 2A. When fully constructed, the disc portion 240 is held between flexible membrane 230 and the opening 245 of the screw attachment portion 110. That is, the diameter of disc portion 240 is larger than the diameter of opening 245. Notably, the flexible membrane 220 may be designed to fit within the cavity formed when rod insertion portion 115 is joined to screw attachment portion 110. In addition, the cavity may be configured to fit tightly when membranes 225 and 230 are also included.

One benefit to the construct of FIG. 2A is that different flexibility levels may be achieved by simply using interchangeable flexible membranes. As shown in FIG. 3, each element may be identical to the elements of FIG. 2A except for the flexible membrane 320 (which replaces flexible member 220). Here, the flexible spinal fixation assembly 300 may utilize a stiffer, less flexible membrane 320. When compared to the flexible membrane 220 of FIG. 2 (and enlarged in FIG. 4), flexible membrane 320 may have four points of attachment 340 to join inner circumference 335 with the frame 350 whereas flexible membrane 220 has only two points of attachment 440 to join inner circumference 235 to the frame 450 as shown in FIG. 4. By flexible membrane geometry configuration or utilizing more points of attachments, the flexible member 320 of FIG. 3 may more firmly hold the position of the inner circumference, and as a result, reduce elasticity and displacement of head component 150. In one aspect, flexible membrane 225 and/or 230 may also be removed or replaced with inflexible members (not shown) to reduce flexibility and prevent the flexible spinal fixation assembly 300 from compressing or elongating along the screw axis when a force is exerted substantially along the direction of the screw axis on the spinal fixation assembly 300.

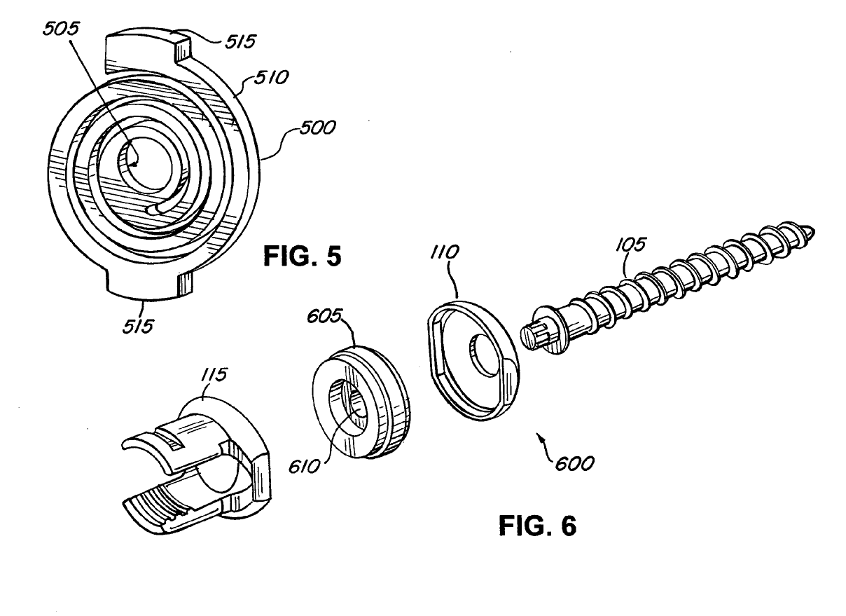

Turning to FIG. 4, the flexible membrane 220 is shown enlarged for clarity. In one aspect, the flexible membrane 220 may be constructed out of a polymer. However, other materials may be used to construct flexible membrane 220 depending on the level of flexibility desired such as polyetheretherketone (PEEK), polycarbonate urethane (PCU), polyester and polyethylene. In addition, the flexible membrane may further incorporate titanium, cobalt chromium, steel and nitinol. Any of the materials discussed herein and derivatives thereof may be mixed-and-matched to obtain the optimal flexibility desired. For example, more rigidity may be achieved if flexible membrane 220 is constructed out of PEEK infused with steel and more flexibility may be achieved if flexible membrane 220 is constructed out of PCU without any metallic alloys. Some embodiments may call for a membrane 220 constructed out of purely metallic alloys. In another aspect, different portions of the flexible membrane (e.g., the frame, attachment elements and the inner circumference) may be constructed out of different materials. In one example, the frame may be desirable with increased stiffness so that PEEK infused with a metallic alloy may be utilized, whereas the attachment portions or elements may be desirable with increased flex so that a polymer without metallic alloys may be utilized. As discussed above, varying degrees of flexibility may also be achieved by altering the design of the flexible membrane. For example, FIG. 5 illustrates another design of a flexible membrane 500. Here, a “swirl-like” pattern is between the inner circumference 505 and the outer frame portion 510. In addition, tabs portion 515 may be included to fit the flexible membrane 500 within a head component. In one aspect, the inner circumference 505 may be said to only have one point of attachment to frame portion 510 and thus flexible membrane 500 may be more flexible than the flexible membrane 220 of FIG. 4 and flexible membrane 320 of FIG. 3, given the same construction material (e.g., a polymer). Various shapes and/or geometries may be used for the flexible membrane depending on the amount of flexibility desired or energy absorption needed.

FIG. 6 illustrates another embodiment of a flexible spinal fixation assembly 600. Here, membrane 605 may be constructed out of flexible material or another polymer is incorporated without other flexible membranes. The assembly 600 of FIG. 6 may provide for less radial displacement than the other embodiments (e.g, assembly 100 of FIG. 2 or assembly 300 of FIG. 3) mainly due to the more rigidly designed membrane 605 and the lack of other flexible membranes. In one aspect, by reducing the thickness of the membrane 605, the cavity formed within the head component may be reduced (i.e., the smaller the membrane, the smaller the cavity required to hold the membrane) and thereby the entire profile or size of the assembly (e.g., assembly 600) may be achieved. A smaller size of the assembly is further advantageous because the pedicle region is not large so smaller assemblies are beneficial in being able to be fit into the pedicle bone.

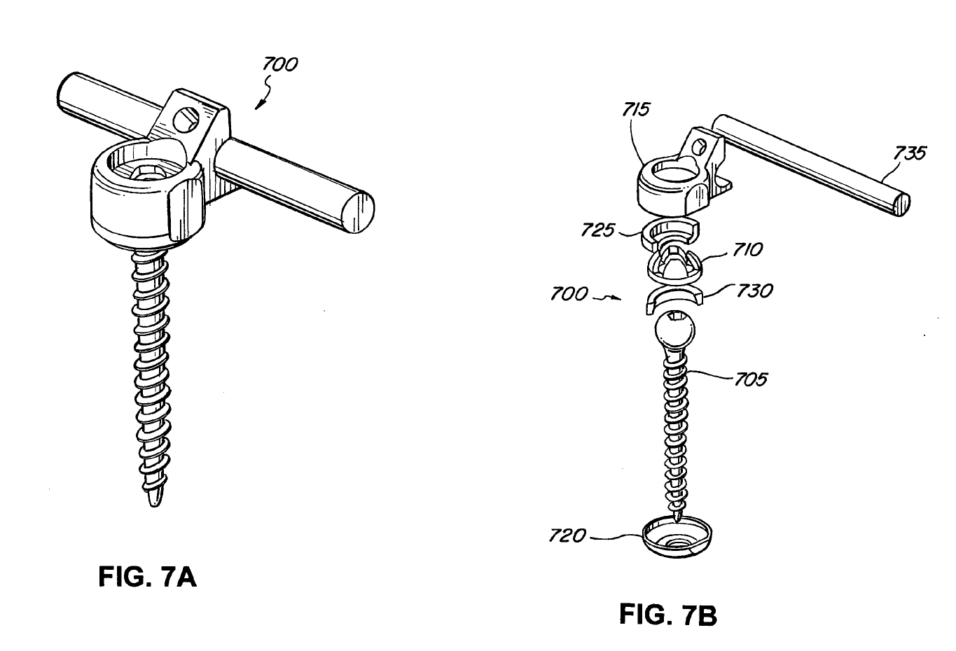

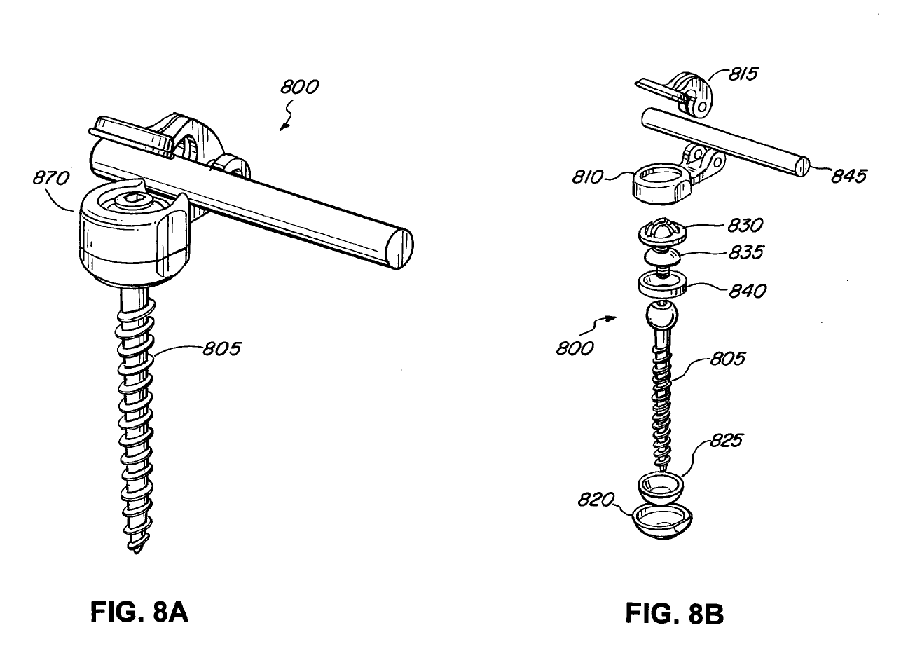

While the assemblies discussed up to this point have been pseudo-polyscrew systems, other screw systems are compatible with the features described herein. For instance, two different embodiments of side-loading polyscrew systems are shown in FIGS. 7A and 8A. FIG. 7A illustrates a side-loading polyscrew assembly 700. FIG. 7B is an exploded view of the side-loading polyscrew assembly 700 of FIG. 7A. As shown, assembly 700 includes a screw 705, a cap 710, rod insertion portion 715, bottom plate 720, and flexible membranes 725 and 730. The assembly 700 may also include a rod 735 for attaching to other assemblies. For example, the rod 735 may attach to another assembly 700 or may attach to other assemblies such as assembly 100 of FIG. 1A. The flexible membrane 725 may be designed to fit between cap 170 and the interior of rod insertion portion 715. The head component (e.g., including the rod insertion portion 715, and the bottom plate 720) may be angulated and rotated to proper position. For example, the screw 705 may be rotated inside membranes 725 and 730 before being locked into location by the attachment of the bottom plate 720 to the rod insertion portion 715. However, even after angulation, positioning and locking, the flexible membrane 725 and 730 may further provide restrictive motion under loading. Flexible membrane 725 may function to provide flexibility to assembly 700 so that the assembly may compress along an axis of the screw 705. Flexible membrane 730 may be designed to fit between the rounded head of screw 705 and the cap 710 and may function to provide flexibility to allow radial displacement of the screw head (comprising of rod insertion portion 715, bottom plate 720, flexible membranes 725 and 730, and cap 710) with respect to the screw axis. In one aspect, the design of flexible membrane 730 may be a “half circle”. Accordingly, this design provides the advantage of allowing the screw head to displace in a direction away from the flexible membrane 730 and preventing the screw head from displacing in a direction towards or proximal to the flexible membrane 730, thereby limiting the direction of flexibility. The design of flexible membranes 730 and 725 may be advantageous in situations where the physician desires to limit the direction of movement of the pedicle attached to the screw.

FIG. 8A illustrates another embodiment of a side-loading polyscrew assembly, namely assembly 800. FIG. 8B is an exploded view of the side-loading polyscrew assembly 800 of FIG. 8A. Assembly 800 may include a screw 805, a bottom rod insertion portion 810, a top rod insertion portion 815, a bottom plate 820, a first flexible membrane 825, a cap 830, a screw seating 835, a second flexible membrane 840. The assembly 800 may also include a rod 845 for attaching to other assemblies. For example, the rod may attach to another assembly 800 or may attach to other assemblies such as assembly 100 of FIG. 1A. As shown, the second flexible membrane 840 may be held in place by the screw seating 835 being threaded into the head of screw 805. The first flexible membrane 825 may include a hole to receive the screw portion of screw 805 and contact the bottom of the head of screw. That is, the hole of 825 may allow the narrower portion of screw 805 to fit through the hole while preventing the head from slipping through. By “cupping” the bottom of the head of screw 805, and having a hole of a size larger than the diameter of the narrower portion, the head component (e.g., 870 of FIG. 8A) may be able to displace and flex angularly with respect to the screw axis, albeit in a limited fashion. The screw 805 may further be fixed by the cap 830. The bottom rod insertion portion 810 and top rod insertion portion 815 may be a hinged arm construct and may be snapped or otherwise brought together and tightened to hold the rod in place. Here, in this embodiment, the head component (e.g., the bottom rod insertion portion 810, and the bottom plate 820) may be angulated and rotated to proper position before being the side-loading polyscrew assembly 800 is tightened. For example, the screw 805 may be rotated inside membranes 825 and 840 before being locked into location by screw seating 835. However, even after angulation, positioning and locking, the flexible membrane 825 and 840 may further provide restrictive motion under loading. When constructed, the entire assembly 800 is as shown in FIG. 8A.

In one aspect, the head component (e.g., head component 150 of FIG. 1 comprising a screw attachment portion 110 and rod insertion portion 115 or rod insertion portion 715 and bottom plate 720 of FIG. 7) may be constructed out of two separate pieces and welded or otherwise attached together. Alternatively, the head component may be one piece molded over the other components. In another aspect, the flexible membranes discussed herein as discussed may be designed to be of different flexibilities and may take different geometries or shapes. Additionally, the flexible membranes may be washer-like devices, springs, spring-like devices and the like.

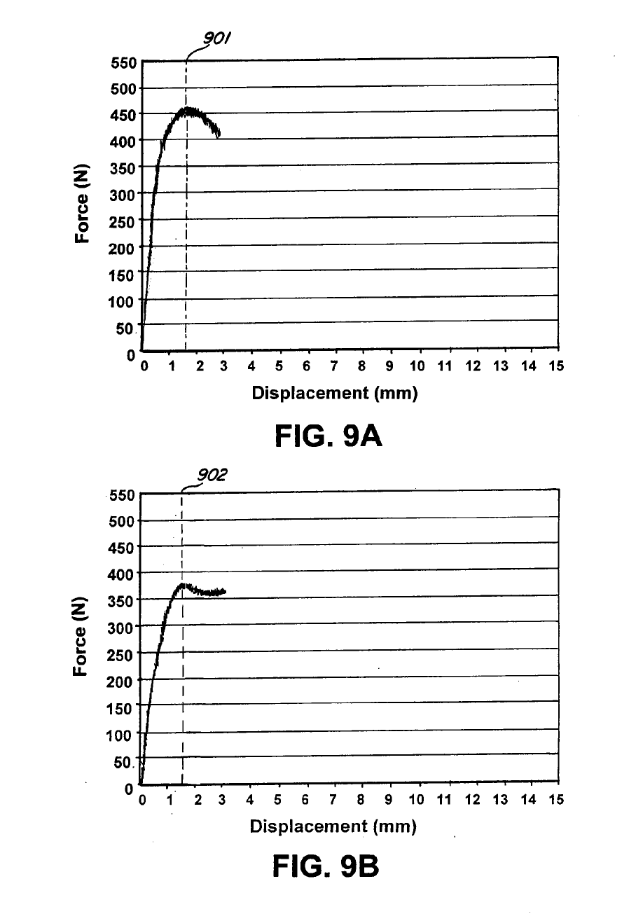

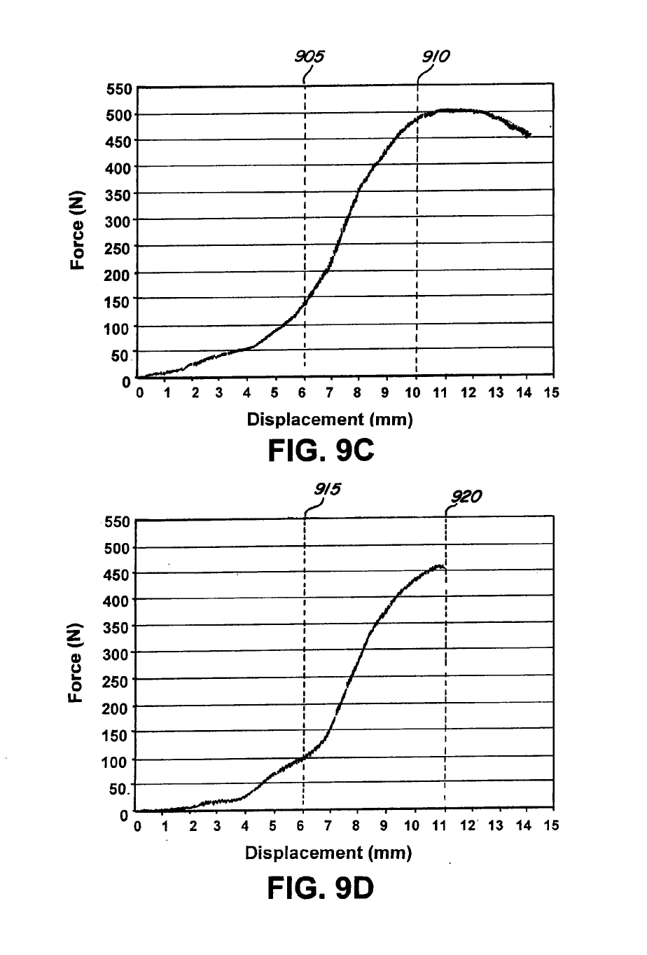

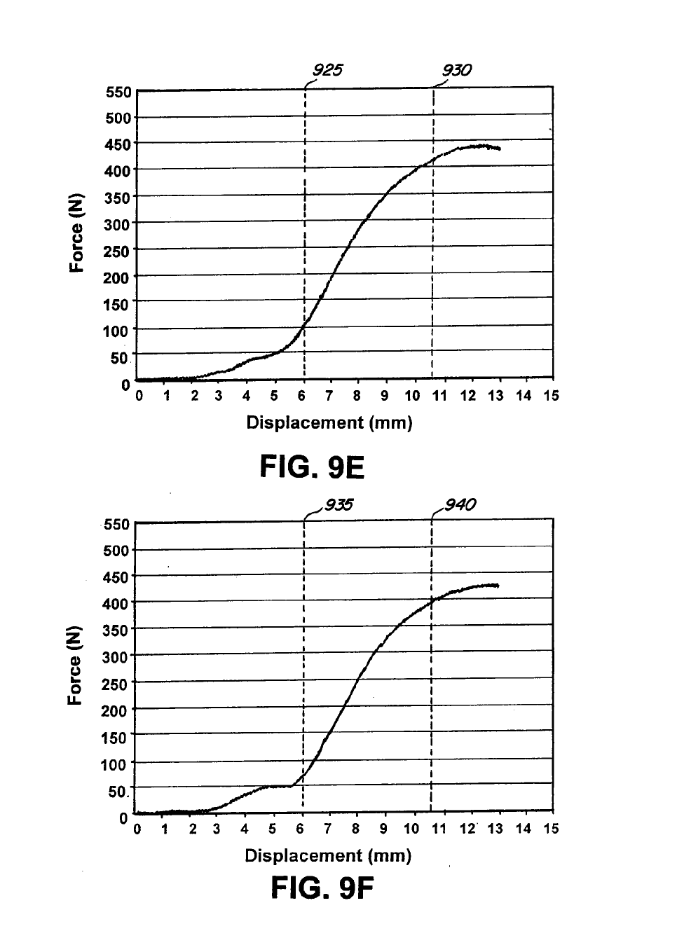

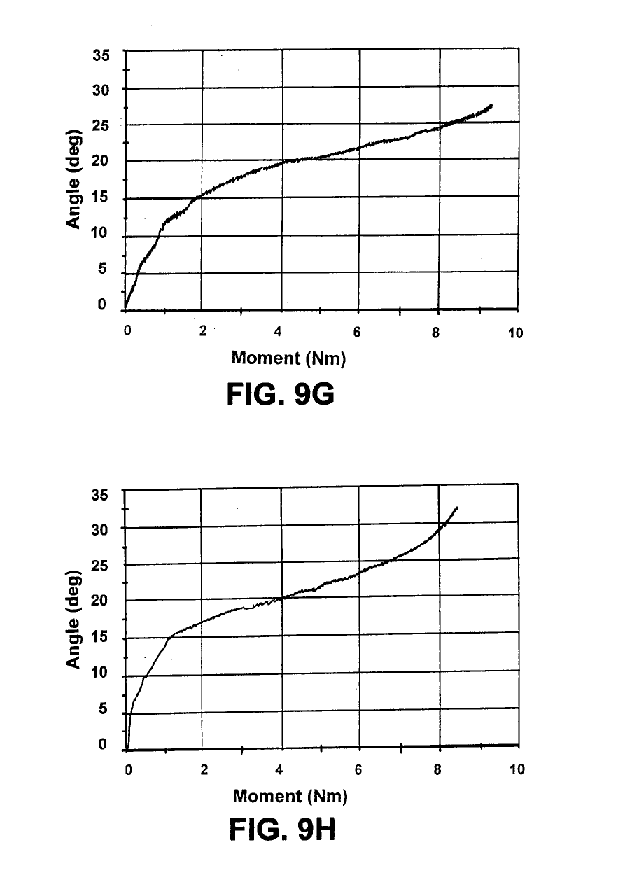

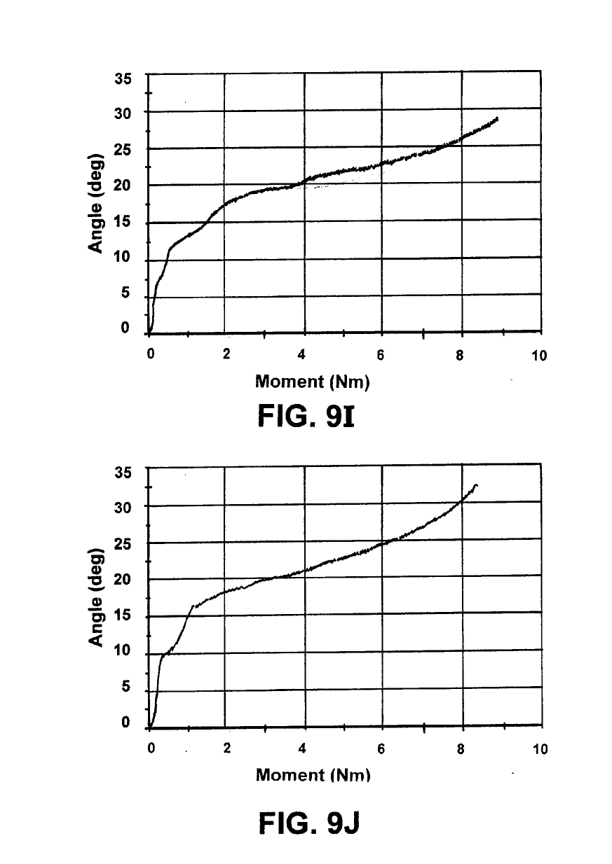

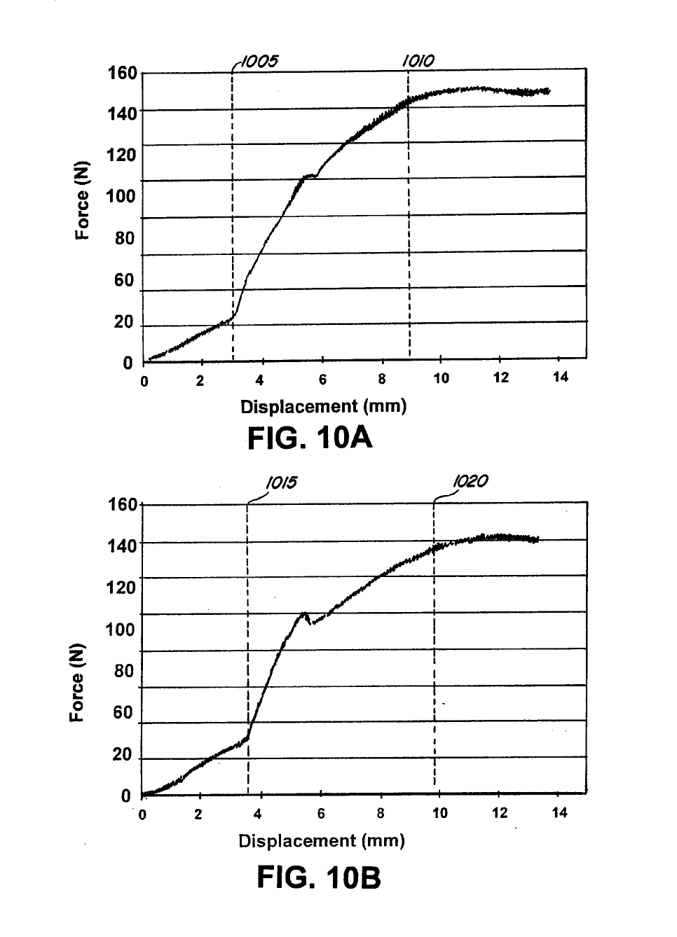

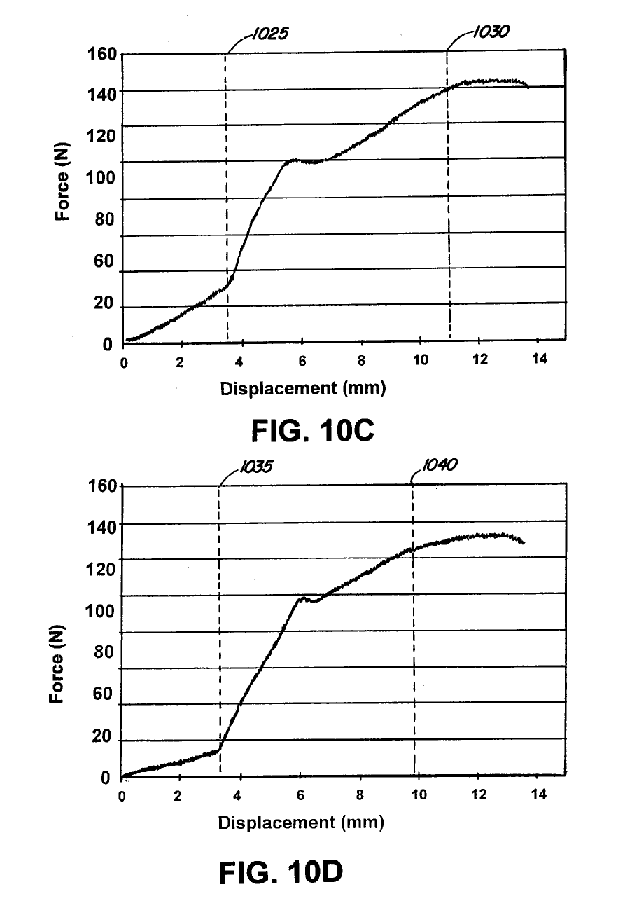

The construction of the various embodiments having been discussed, the results and net effect of these embodiments will now be described. As described above, for the sake of clarity and simplicity, the motion of flexibility has been described with respect to an axis defined by the screw (and as such, describing the displacement of the head component). However, by fixing the position of the head component and using that as the point of reference, the displacement of the screw tip may be measured and also described. As such, the point of reference is of less importance than the true measure of difference between the position of the head component and the screw. Of further importance is that the flexible spinal fixation assemblies described herein are configured to be able to provide movement of the head component in at least three directions. First, flexion-extension movement (e.g., moving away from the screw axis or in other words, movement, flexing or bending of the head component with respect to the screw axis), second, torsion movement (twisting about the screw axis or in other words, the rotation of the head component about any position obtained in the flexion-extension movement), and third, slight compression or extension of the flexible spinal fixation assembly. FIGS. 9A-9J describe flexion-extension movement, while FIGS. 10A-10D describe torsion movement.

Turning now to FIGS. 9A and 9B, these figures illustrate displacement-force graphs for prior art systems. As shown in FIG. 9A, the distance of displacement of the screw tip during load tests when the head component is fixed is shown to be minimal. By applying a force of 450 newtons (N), the screw tip for a traditional assembly only displaces between 1 and 2 millimeters (mm) before failing (the graph shows failure at about 2 mm, hence the appearance of increased displacement at a force of 400 newtons). The dotted line 901 illustrates the point of failure for the prior art system of FIG. 9A. Similarly, FIG. 9B illustrates a displacement of between 1 and 2 millimeters prior to failure as shown by dotted line 902. In one aspect, the prior art systems of FIGS. 9A and 9B depicted here allow for very limited flexibility and may be deemed “one stage” as slope of the force-displacement graph is constant until failure.

FIGS. 9C-9F illustrate sample displacements of the different embodiments described herein. As shown in FIG. 9C for example, the screw tip greatly displaces (from 0 mm to 6 mm) by applying a force of only 150 N. At the threshold depicted by dotted line 905, the force-displacement relationship changes. To displace the screw tip an additional 4 mm, a force of over 450 N is needed. In other words, one or more assemblies described herein have a two-stage displacement, a first stage shown to the left of dotted line 905 having great displacement requiring only a small force, but then transitioning into a second stage to the right of dotted line 905 when a threshold of force is exceeded. The second stage is characterized by a result where flexibility is greatly reduced and for further displacement a proportionally larger force is needed. Advantageously, the patient may be allowed small movement (e.g., up to 6 millimeters) without much resistance, but greater movement may need a greater force from the patient, thereby preventing “accidental” greater movements and preventing bone degradation and cracking while maintaining sufficient rigidity to allow healing. It may be said that the behavior of the assembly is such that the assembly shows a controlled flexible motion up to a certain point (the threshold), and behaves as a more rigid construct thereafter. The advantageous two-stage displacement feature is based upon the advantageous design of the flexible membrane within the spinal assembly construct, and also based upon the materials used to construct the membrane. Dotted line 910 illustrates a point of failure as another point of comparison to the prior art systems (e.g., comparing dotted line 910 of FIG. 9C and dotted lines 901 and 902 of FIGS. 9A and 9B, respectively).

FIGS. 9D, 9E and 9F illustrate other two stage assemblies with different examples of transition forces and displacements. As shown, prior to thresholds demarked by lines 915 and 925 of FIGS. 9D and 9E, respectively, little force (roughly 100 N) is required to displace the screw tip to about 6 mm, then to displace the tip 4 mm more, an additional force of about 300 N may be needed. Beyond the 10 mm point, the assembly fails as shown by dotted lines 920 and 930 of FIGS. 9D and 9E, respectively. FIG. 9F illustrates a similar characteristic. Here, a small amount of force (about 50 N is needed to displace the screw tip 6 mm as shown by dotted line 935. However, an additional 350 N is needed to displace the screw tip an additional 4-5 mm (before failure, shown as dotted line 940).

FIGS. 9G-9J correspond with FIGS. 9C-9F, respectively. Instead of illustrating a force-displacement relationship, FIGS. 9G-9J illustrate an angle-moment relationship. That is, FIGS. 9G-9J illustrate the angle of displacement as measured between the screw tip and the head component for the particular moments. For example, as shown in FIG. 9G, for a torque of roughly 2 newton-meters, the screw tip displaces roughly 17 degrees away from the head component. However, to obtain a further displacement of 10 degrees, an additional 7 newton-meters is required. This is consistent with FIG. 9C. Similarly, FIGS. 9H-9J exhibit results analogous to the data shown in FIGS. 9D-9F. As an example, FIGS. 9C-9J may be obtained by using a flexible membrane such as membrane 320 of FIG. 3, possibly with each of FIGS. 9C-9F (and corresponding FIGS. 9G-9J) utilizing a different polymer with slightly different flexibilities.

FIGS. 10A-10D illustrate four torsion force-displacement graphs corresponding to the four assemblies of FIGS. 9C-9F, respectively. Here, FIGS. 10A-10D show displacements of 10 or more millimeters resulting from a force of under 160 N. Similar to the FIGS. 9C-9F, FIGS. 10A-10 D illustrate a two stage torsion force-displacement. For example, as shown in FIG. 10A, prior to threshold 1005, a small amount of force (˜20 N) is needed to rotate the head component 3 mm, but a proportionally larger force (˜120 N) is needed to rotate it an additional 6 mm before the assembly fails at the threshold 1010. FIGS. 10B, 10C and 10D illustrate a similar characteristic. Dotted lines 1015, 1025 and 1035 of FIGS. 10B, 10C and 10D, respectively, illustrate the threshold where the force required for further displacement substantially increases. Accordingly, dotted lines 1020, 1030 and 1040 of FIGS. 10B, 10C and 10D, respectively, illustrate the threshold where the assemblies fail.

The terms “a,” “an,” “the” and similar referents used in the context of describing the invention(especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references may have been made to patents and printed publications in this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1-21. (canceled)

22. A spinal fixation device comprising:
a screw, the screw comprising a screw head and comprising a screw shaft fixable to a patient's spine and having a screw axis;
a head component having an interior cavity and having an opening connecting the interior cavity to an exterior, the opening being larger than a corresponding diameter of the screw head, such that the screw head can pass through the opening; and
a first deformable member housed inside the head component,
wherein the first deformable member allows displacement of the head component by elastic deformation of the first deformable member so that, with respect to the screw, the head component can move in a radial direction away from the screw axis.

23. The spinal fixation device of claim 22, wherein the first deformable member comprises an inner circumference and an outer frame and at least a first curved member between the inner circumference and the outer frame, and wherein the screw head is contained inside the inner circumference.

24. The spinal fixation device of claim 23, wherein the first curved member connects to the outer frame at a first outer connection point and connects to the inner circumference at a first inner connection point, and further comprising a second curved member that connects to the outer frame at a second outer connection point and connects to the inner circumference at a second inner connection point.

25. The spinal fixation device of claim 23, wherein the first curved member forms a spiral extending between the inner circumference and the outer frame.

26. The spinal fixation device of claim 23, wherein during the displacement in the radial direction, the displacement in the radial direction away from the screw axis causes the inner circumference to move closer to or farther away from the outer frame with respect to a particular direction.

27. The spinal fixation device of claim 22, wherein the first deformable member has an outer shape that is non-circular whereby, with respect to rotation around the screw axis, the first deformable member is maintained in a fixed position with respect to the head component.

28. The spinal fixation device of claim 22, further comprising a second deformable member housed inside the head component, wherein the second deformable member has an axial-facing surface in contact with the first deformable member, and wherein the second deformable member is configured to allow compression or elongation motion of the head component, with respect to the screw, along the screw axis.

29. The spinal fixation device of claim 28, wherein the second deformable member has a shape of a washer.

30. The spinal fixation device of claim 28, wherein a stiffness for the displacement of the head component relative to the screw in the radial direction is different from a stiffness for the elongation or compression motion of the head component relative to the screw along the screw axis.

31. The spinal fixation device of claim 22, wherein the first deformable member is configured to allow motion in a torsional degree of freedom of motion, wherein the torsional degree of freedom is rotation of the head component about the screw axis with respect to the screw.

32. The spinal fixation device of claim 22, wherein the first deformable member is configured to allow motion in a tilting degree of freedom of motion, wherein the tilting degree of freedom is tilting of the head component with respect to the screw.

33. The spinal fixation device of claim 32, wherein tilting of the head component relative to the screw is such that there is a first moment-angle slope in a low-moment region and there is a second moment-angle slope in a high-moment region, the first moment-angle slope and the second moment-angle slope being different from each other.

34. The spinal fixation device of claim 22, wherein displacement of the head component relative to the screw is such that there is a first force-displacement slope in a low-force region and there is a second force-displacement slope in a high-force region, the first force-displacement slope and the second force-displacement slope being different from each other.

35. The spinal fixation device of claim 22, wherein the head component accepts a spinal rod through a top-oriented opening or through a side-oriented opening.

36. An assembly comprising two of the spinal fixation devices of claim 22, wherein a first of said spinal fixation devices comprises a first device first deformable member, and a second of the spinal fixation devices comprises a second device first deformable member, the two devices being connected to a common spinal rod, wherein the first device first deformable member and the second device first deformable member have different stiffnesses with respect to at least one direction of motion.
